(19) United States
(12) Patent Application Publication
(10) Pub. No.: US 2021/0038637 A1
(43) Pub. Date: Feb. 11, 2021

(54) ANTIBACTERIAL AND SPERMICIDAL LUBRICANT

(71) Applicant: BCSK Biocid GmbH, Vienna (AT)

(72) Inventor: Lukas Mayerhofer, Vienna (AT)

(73) Assignee: BCSK Biocid GmbH, Vienna (AT)

(21) Appl. No.: 16/966,793

(22) PCT Filed: Feb. 8, 2019
(86) PCT No.: PCT/EP2019/053127

§ 371 Date: Jul. 31, 2020

(30) Foreign Application Priority Data
Feb. 8, 2018 (EP) 18155747.1

(51) Int. Cl. A61K 31/785; A61P 15/18; A61P 15/00; A61K 9/00; A61K 31/765

(52) U.S. Cl. CPC A61K 31/785; A61P 15/18; A61K 31/765; A61K 9/0034; A61P 15/00

(57) ABSTRACT

The invention relates to a chemical composition, which contains at least one polymeric guanidine biocide and at least one alkylphenoxy polyethoxyethanol spermicide in an aqueous solution, and at least one thickener, and which is suitable as a biocidal and spermicidal agent for use during intercourse.

The present invention relates to intimate use of a spermicide in a stable gel formulation.

Intimate formulations with a spermicide have been described in the literature. Thus, U.S. Pat. No. 5,512,289 describes a formulation with an alkylphenoxypolyethoxyethanol spermicide such as, for example, the usual spermicide nonoxynol-9, and a solubiliser from the group formed by non-ionic polyethoxylated compounds, primarily polyethylene glycol (PEG) or polysorbate (Tween). Large quantities of such solubilisers, above all of PEG, are essential, because otherwise, the spermicide precipitates out and milky pastes are formed. PEG and polysorbate are harmful substances because they reduce the barrier function of the skin. This is particularly unsuitable in the case of an intimate formulation which is additionally supposed to reduce the risk of infections.

Alkylphenoxypolyethoxyethanols, in particular nonoxynol-9, are themselves strong surface active substances (U.S. Pat. No. 6,028,115) and it is therefore detrimental to their action if microphase separations such as emulsions occur in a formulation, because this gives rise to microheterogeneities in the formulation, forming zones of different activities, including severely weakened activities.

One objective of the present invention is to provide alternative formulations with an alkylphenoxypolyethoxyethanol spermicide which can dispense with the addition. of polymeric compounds such as PEG or polysorbate. A further objective is the provision of a biocidal substance in a lubricant in order to reduce the risk of infection with a sexually transmitted disease.

Both objectives are achieved in accordance with the invention by means of the use of polymeric guanidines as the biocidal substances which in addition, surprisingly, permit stable thickened solutions to be formed in lubricant gels. “Stable” as used herein means that a phase separation, such as that observed in the comparative examples of U.S. Pat. No. 5,512,289, is avoided.

WO 2008/031105 A1 describes polymeric guanidine germicides for the topical treatment of animal skin.

UA 64 406 A (abstract) describes an ointment with polyhexamethylene guanidine phosphate and various oils for the treatment of necrotic ulcerative gingivostomatitis.

Surprisingly, it has been shown that PEG, polysorbate or other solubilisers from the group of non-ionic polyethoxylated compounds can be dispensed with when the antimicrobial substance from the polymeric guanidine group is added to the formulation in small quantities.

The invention therefore concerns a chemical composition containing at least one polymeric guanidine biocide and at least one alkylphenoxypolyethoxyethanol spermicide, in aqueous solution, and at least one thickening agent.

The composition enables the transmission of sexually transmitted diseases during sexual activity to be prevented and simultaneously provides contraception without the use of a mechanical protective. The use of a gel formulation offers the advantages of a lubricant gel in order to allow for painless and smoother sexual intercourse. This stops the appearance of microlesions which occur during sexual activity without lubricant, constituting an additional risk of infection. In this regard, it has surprisingly been shown that the biocidal action of the polymeric guanidine biocide is reinforced by the composition in accordance with the invention.

“Prevention” or in fact “inhibition” should not be understood to mean absolute effects, i.e. as a 100% successful prevention, but as a reduction in the risk or probability of transmission of sexually transmitted diseases and/or of conception.

Polymeric guanidine biocides are known in the specialist field of disinfectants. Their manufacture has been described in WO 01/85676 A1 and in the patents AT 408302 B and AT 411060 B. Polymeric guanidine biocides are also known as Akacid, or X-Cid, and have been described in Kratzer et al., Antibiotika Monitor Jan. 2, 2006; Buxbaum et al., Journal of Antimicrobial Chemotherapy (2006) 58,193-197; U.S. Pat. No. 2,325,586; GB 1 095 902 A; WO 1999/054291 A1, WO 01/85676 A1; WO 2002/030877 A1; WO 2006/047800 A1; WO 2008/080184 A2; WO 2009/092123 A2; EP 2 520 605 A1; WO 2013/064161 A1; WO 2014/113835 A1; WO 2016/015081 A1 (all incorporated herein by reference). The polymeric guanidine biocide may also be in the form of a complex, for example with gelatine or a polysaccharide (for example as described in WO 2010/106007 A1). WO 2008/080184 describes the use of polymeric guanidines to control microorganisms in non-therapeutic applications, for example by nebulization for the disinfection. of spaces. A composition which is mentioned in this regard is Akacid, poly-[2-(2-ethoxy)ethoxyethyl]guanidinium chloride and Akacid Plus, a 3:1 mixture of poly (hexamethylene guanidinium chloride) and poly[2-(2-ethoxy)ethoxyethyl]guanidinium chloride].

In this regard, the term. “polymeric guanidine” in particular is used to represent. “polymeric guanidine derivatives based on an alkylene diamine and/or an oxyalkylene diamine” (WO 2009/009815 A1), in particular for “biocidal polymeric guanidine derivatives based on diamines which contain alkyl chains or oxyalkyl chains between two amino groups, wherein the guanidine derivatives are a product of polycondensation of a guanidine-acid addition salt with diamines which contain polyalkylene chains or polyoxyalkylene chains between two amino groups” (EP 1 280 766 B1, claim 1). A poly(hexamethylene guanidinium) salt and/or poly[2-(2-ethoxy)ethoxyethyl]guanidinium salts are particularly preferred. Akacid and Akacid Plus are preferred polymeric guanidine biocides in accordance with the present invention.

A preferred embodiment of the composition in accordance with the invention is characterized in that a polymeric guanidine biocide is provided which contains an alkylenediamine and the oxyalkylenediamine in a molar ratio between 4:1 and 1:4. The amino groups of the alkylenethamine and/or of the oxyalkylenediamine are preferably terminal, wherein in order to produce the polymeric guanidine biocide, initially, as the alkylenediamine, a compound with the general formula NH₂(CH₂)ₙNH₂ is provided, in which n is a whole number between 2 and 10, in particular 6. In order to produce the polymeric guanidine biocide, as the oxyalkylenediamine, a compound with the general formula NH₂[(CH₂)₂O)]ₘ(CH₂)₂NH₂ is provided, in which n is a whole number between 2 and 5, in particular 2.

Preferred polymeric guanidine biocides may be selected from poly(hexamethylene guanidine; poly[2-(2-ethoxy)-ethoxyethyl]guanidine; polytriethyleneglycol guanidine; polyethyleneglycol guanidine; polyoxypropylene guanidine; polyoxyethylene guanidine.

Particularly preferably, the polymeric guanidine biocide is a polyalkylene guanidine, in particular a polyoxyalkylene guanidine. “Alkylene” may be a C1-C8 alkyl, most preferably a C2-C6 alkyl such as ethyl, propyl, butyl, methylpropyl, pentyl, which may or may not be branched. This is preferred in all uses herein of the term “alkylene” or “alkyl”.

Further preferred polymeric guanidines which are extremely suitable for the present invention are described in WO 2014/113835 A1 and WO 2016/015081 A1 (both incorporated herein by reference). Polymeric guanidines of this type. (also known as “polyguanidines”) may correspond to formulae (I), (II) or (III):

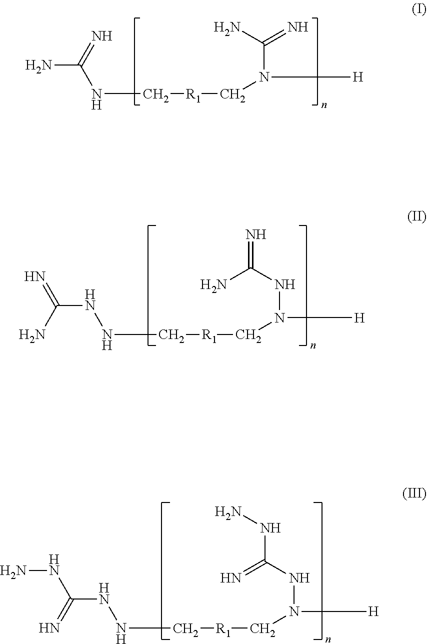

wherein R₁ represents either an aromatic ring system containing at least one aromatic ring which optionally contains one or more heteroatoms selected from O, N and S and which is optionally substituted with one or more vinyl groups, or it represents ethylene, or it has a cyclic structure obtained by ring closure with the elimination of a guanidine. Examples of R₁ are benzene (preferably bonded in the para- or meta-position), pyridine (preferably bonded to the two C atoms adjacent to the N), divinylbenzene, biphenyl (preferably respectively both benzenes bonded in the para-position), 1,3-bis((E)-2-vinyl) benzene, furan, pyrrole, thiophene, fluorene, ethylene (preferably in the cis-conformation). Polymeric guanidines of this type have been described in WO 2016/015081 A1.

Polymeric guanidines may also contain the formula (IV)

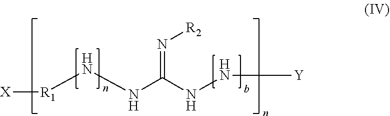

wherein

X ls selected from —NH₂, aminoguanidino and 1,3-diaminoguanidino;

Y is selected from —H and —R₁—NH₂; or X and Y together represent a chemical bond for the production of a cyclic structure;

R₁ is selected from divalent organic residues containing 2 to 20 carbon atoms in which optionally, one or more carbon atoms are replaced by O or N;

a and b are respectively 0 or 1;

wherein a+b do not equal 2 when no 1,3-diaminoguanidine units are present;

R₂ is selected from —H and —NH₂;

wherein R₂ is —NH₂ when a+b=0;

R₂ is —H or —NH₂ when a+b=1; and

R₂ is —H when a+b=2; and n is more than 2;

or a salt thereof. Polymeric guanidines of this type are described in WO 2014/113835 A1,

In formulae (I) to (IV), n corresponds to a multiple, for example in order to reach the molar mass of the polymer defined above. As an example, n may be 3 to 200.

The polymeric guanidines in the examples of WO 2014/113835 A1 and WO 2016/015081 A1 are particularly preferred (examples from both publications incorporated herein by reference). The polymeric guanidine polymetaxylidene aminoguanidine (PMAG), particularly preferred in all aspects of the present invention.

The polymeric guanidines of WO 2014/113835 A1 and WO 2016/015081 A1, particular PMAG, have the advantages that they can be manufactured with small amounts of residual monomers (such as HMDA—hexamethylenediamine—which is often present) and therefore exhibit low toxicity, which is of particular advantage in the context of the invention. A. composition formed by a gel forming agent (or another thickening agent), nonoxynol (or another spermicide), PMAG (or another polymeric guanidine, in particular from WO 2014/113835 A1 or from WO 2016/015081 A1) and optionally lidocaine (or another local anesthetic) is particularly preferred.

Preferably, the guanidine is a guanidinium salt, preferably selected from a halide, preferably a chloride; phosphate, preferably a dihydrogen phosphate; carbonate; nitrate; sorbate; acetate, preferably hydroacetate; gluconate, citrate, silicate; etc. This is also the case for polymeric guanidine. The polymeric guanidine in accordance with the invention may be a polyguanidine salt. These compounds with salt-forming partners (counter-ions), such as halide, phosphate, etc, for example, are described in AT 411060 B.

Preferably, the mean molecular weight of the polymeric guanidine biocide is 200 Da to 10000 Da, preferably 500 Da to 3000 Da. As an example, the polymeric guanidine biocide may be provided with at least 3 guanidine residues. The polymers with these sizes may be obtained by nanofiltration through membranes. For nanofiltration, filters with appropriate pore sizes to allow the desired molar masses to pass through are used. Filtrates (for removing large polymers) or the residue (for removing the smaller polymers) may be recycled in order to obtain the desired polymer. The desired, preferably polymeric guanidine biocides have an improved toxicology. In particular, the elimination of short-chain oligomers and monomers, as well as the elimination of the residues of the diamines entrained by the synthesis is a major advantage. These could otherwise be resorbed by the vaginal lining, which could have unwanted pharmaceutical effects. The major advantage of the formulation here is the local application and three-fold action without unwanted side effects. Correspondingly, monomeric starting materials (guanidines) and oligomers or polymers of the guanidine biocide with a molecular weight of 250 Da or less, or even of 400 Da or less, are removed.

The proportion of monomers and short-chain oligomers of guanidine with a molecular weight of 250 Da or less, such as HMDA, is preferably less than 0.1% by weight of the composition, particularly preferably less than 0.05%, particularly preferably less than 0.01%, less than 0.005%, less than 0.001% or even less than 0.0001% of the composition (all as a % by weight). Particularly preferably, the composition is free from them, or they are removed as described above, for example by nanofiltration.

The polymeric guanidine biocide is provided in a sufficient quantity or concentration to inhibit the growth of pathogenic bacteria (gram negative and/or gram positive), which appear during sexual intercourse. Examples of bacteria of this type which are inhibited in accordance with the invention are S. aureus, E. coli, P. aeruginosa.

As an alternative or in combination, the polymeric guanidine biocide may be provided in a sufficient quantity or concentration to inhibit the growth of pathogenic fungi which appear during sexual intercourse. Examples of fungi of this type which are inhibited in accordance with the invention are C. aibicans, S. cerevisiae.

As an alternative or in combination, the polymeric guanidine biocide may be provided in a sufficient quantity or concentration to inhibit the transmission and infection of the receiving sexual partner with viruses which appear during sexual intercourse. The biocidal action of the polymeric guanidine biocide counters these pathogens, primarily bacteria. As far as possible, the host or the sexual partner (in particular human beings) should not be compromised by the polymeric guanidine biocide. Human beings are the preferred users of the composition in accordance with the invention who are to be treated.

The composition in accordance with the invention may furthermore contain an alkylphenoxypolyethoxyethanol spermicide such as nonylphenoxypoly (ethyleneoxy) ethanol (nonoxynol-9). This is particularly preferable for use as a “liquid condom”, i.e. an agent which is liquid, fluid. or fluidizable upon use (i.e. “liquid”) for contraception, additionally for the prevention of the transmission of diseases. Preferred spermicides which may be used in accordance with the invention are nonoxynol-9, octoxynol-9, dodecaethyleneglycol monolaurate, laureth 10S and methoxypolyoxyethyleneglycol 550 laurate.

For particular applications, namely for use by homosexuals, this spermicide is not necessary. Instead, in this case, a local anaesthetic such as lidocaine is used in order to delay a premature orgasm during sexual intercourse, in particular between men. A local anesthetic of this type may also be used during heterosexual sexual intercourse, but here it is less preferred because of the effect on the female and here too, the local anesthetic could be dispensed with. Preferred other local anesthetics may include benzocaine, dibucaine, benzyl alcohol, camphor, resorcinol, menthol and diphenylhydramine hydrochloride and the like, although this is not limiting.

Alkylphenoxypolyethoxyethanol spermicides such as nonoxynol-9 are known and have been described in U.S. Pat. No. 5,512,289, for example. As mentioned above in the introduction, the combination of an alkylphenoxypoiyethoxyethanol spermicide with an aqueous polymeric gel matrix gives rise to problems with dissolution, wherein U.S. Pat. No. 5,512,289 proposed surface active substances such as polysorbate or large quantities of PEG. In accordance with the invention, these can be dispensed with, because surprisingly, it has been shown that the alkylphenoxvpoiyethoxyethanol and the polymeric guanidine biocide have opposing solubilizing roles, and because of this synergistic action are particularly suitable for a composition for sexual intercourse. In accordance with the invention, both substances (the polymeric guanidine biocide and the aikylphenoxypoiyethoxyethanol) are present in solutions or can be dissolved, and in fact completely without phase separation or the formation of a cloudy emulsion because of this chemical interaction.

In particular, the composition in accordance with the invention is a lubricant gel or lubricant for sexual intercourse.

These lubricant gels or lubricants have an enhanced viscosity which is obtained by means of a thickening agent. The viscosity may be 1 Pa-s or more, particularly preferably 1 Pa-s to 900 Pa-s, specially preferably 7 Pa-s to 500 Pa-s, particularly preferably 35 Pa-s to 150 Pa-s. The viscosity is determined at 36° C. and under atmospheric pressure (1 bar).

Preferably, the thickening agent is a gel forming agent. It may, for example, be selected from hydroxyalkyiceiluiose, cellulose, hydroxyethylcellulose, hydroxypropyiceliulose, hydroxypropylmethyicellulose. The thickening agent in suppositories may be a suppository base, preferably selected from hard fat (Adeps Neutralis), a glycerol-gelatine mixture or soluble PEG, for example PEG 400/4000, wherein PEG is less preferred, or is not in the composition in accordance with the invention because of the effect on membranes and an increase in their permeability.

The thickening agent may also have film-forming properties. Thus, in combination with or independently of the thickening agent, the composition may preferably contain one or more film forming polymers, gums, chltosans or the like which, for example, are derived from water-soluble celluloses. Preferably, such polymers derived from cellulose are hydroxyalkylcelluiose polymers. More preferably, the hydroxyalkyicellulose polymer is selected from the following groups: hydroxyethylcellulose, carboxymethylcellulose, hydroxypropylcellulose and hydroxyproylmethlcellulose and the like. Most preferably, the hydroxyalkylcellulose polymer hydroxypropylcellulose. “Alkyl” may be defined as above for polymers (C1-C8 alkyl, including preferred embodiments).

Preferably, the composition contains 0.05% to 1% by weight of polymeric guanidine biocide, particularly preferably 0.1% to 2%, especially preferably 0.4% to 3% (all as a % by weight).

Preferably, the composition contains 0.05% to 8% (by weight) of alkylphenoxypolyethoxyethanol spermicide, particularly preferably 0.1% to 6%, especially preferably 0.3% to 2% (all as a % by weight).

Preferably, the composition contains 1% to 95% (by weight) of thickening agent. In the case of (viscous) liquids and gels, the composition preferably contains 1% to 30% of thickening agent which may be a gel forming agent. Preferably, the range is 1.5% to 20%, particularly preferably 2% to 6% (all as a % by weight). In the case of suppositories, the quantity of thickening agent is preferably 50% to 95%, particularly preferably 80% to 93%.

The composition may be provided in a single dose form, for example in a quantity of 1 mL to 10 mL, preferably 2 mL to 5 mL. An example of a single dose form is a disposable pipette. A disposable pipette may be a plastic pipette the head of which is twisted off before use.

Furthermore, the composition may contain a humectant, for example propylene glycol or glycerine. A humectant may be provided in a concentration of 1% to 30%, preferably 3% to 18% (all as a % by weight), in particular in a liquid or gel composition.

Preferably, the composition is a homogeneous composition (no distinctive phases) which can be administered vaginally as is. In particular, it does not need a solid support such as a sponge and is used without any such solid supports which also remain solid in use (in contrast to a suppository).

Furthermore, the composition may contain other acidification agents such as Acidum citricum or have an additional antioxidant action, such as with Acidum Ascorbicum.

Preferably, the composition has a pH in the range from 4.5 to 8, particularly preferably 5 to 7. The pH may be adjusted using conventional acids and bases and is preferably buffered, i.e. an acid or base should be a weak acid or base. Preferably, acids are selected from carbonic acids, citric acid, phosphoric acid, acetic acid, malic acid, hydrochloric acid, HEPES, etc.

Preferably, only the substances cited here are used and in particular no (other) substances which enhance passage through the epithelial layer such as PEG or polysorbate are used. Solubilisers from the group formed by polyethoxylated non-ionic compounds (these also include polyethylene glycol (PEG) or poiysorbate (Tween)) are also preferably avoided. So that the protected invention according to these embodiments cannot be circumvented easily, small or insignificant quantities of these unwanted substances may be present, for example a maximum of 1% or a maximum of 0.5% (all as a % by weight).

Further unwanted substances are oxidizing substances such as povidone-iodine; these are either completely absent or are only present in small quantities, for example a maximum of 0.5% or a maximum, of 0.1% (all as a % by weight).

Similarly, surface active substances such as benzalkonium chloride are considered to be unwanted substances because they can increase the permeability of the vaginal lining to pathogens. These substances are preferably present in a maximum of 1%, especially preferably a maximum of 0.1% (all as a % by weight). For clarification purposes only, the substances in accordance with the invention, such as the polymeric guanidine biocide or the spermicide or the local anesthetic are not included as unwanted substances.

The composition may also contain pharmaceutical support substances, binders, preferably polymeric binders and/or additives. The term “support substance” refers to a thickening agent, for example water, saline, binder or medium, with which the composition can be administered. With a solid or liquid composition, the support substances or additives in the pharmaceutical composition may be SiO₂, TiO₂, a binder such as, for example, microcrystalline cellulose, polyvinylpyrrolidone (polyvidone or povidone), gum tragacanth, gelatine, starch, lactose or lactose monohydrate, alginic acid, corn starch and the like; a lubricant or surfactant such as magnesium stearate or sodium lauryl sulphate; a flow regulating agent such as colloidal silicon dioxide.

A particularly preferred composition in accordance with the invention contains the following:

polymeric guanidine biocide: 0.05% to 10%,

humectant: 5% to 15%,

alkylphenoxypolyethoxyethanol spermicide: 0.05% to 10%,

thickening agent: 1% to 10%,

all as a % by weight.

The composition may furthermore contain:

Acidum Ascorbicum and/or Acidum Citricum: 0% to 3%,

sodium hydroxide: 0% to 2%,

citric acid: 0% to 4%,

Aqua purificata: remainder to 100%,

all as a % by weight.

Instead of or in addition to sodium hydroxide and citric acid, the composition may also contain buffer components as described above, in particular in order to adjust the pH as described above.

Furthermore, the present invention concerns a use of the composition in accordance with the invention according to any embodiment for vaginal antibacterial, antimycotic and/or antiviral disinfection. The invention also concerns the use thereof as a lubricant during sexual activity, as well as both functions in combination. Because nonoxynol is optional (for example as a pure lubricant without contraceptive action), the invention also concerns the use of a chemical composition containing at least one polymeric guanidine biocide in aqueous solution, and at least one thickening agent for vaginal antibacterlal, antimycotic and/or antiviral disinfection during sexual activity or a method for preventing transmission of a sexually transmitted disease during sexual activity by introducing a composition containing at least one polymeric guanidine biocide in aqueous solution and at least one thickening agent into the sexual organ participating in the sexual activity, in particular into body orifices. Preferably, the composition or one of its components or use is as defined above and below.

Furthermore, the invention concerns a method for preventing infection with a sexually transmittable disease or for contraception during sexual activity, comprising introducing a composition according to any embodiment of the invention into a vaginal canal for the sexual activity.

Mechanical protection such as a sponge or a barrier, by solid objects in the vagina, are not necessary and can be omitted in the use in accordance with the invention.

The composition in accordance with the invention is preferably a lubricant gel in the context of a viscous liquid in which the active substances are dissolved. A lubricant gel has the advantage of painless and smoother sexual intercourse. The dryness of the vaginal lining which principally occurs in post-menopausal women is counteracted by the gel nature of the formulation, whereupon no pain occurs during sexual activity, or the risk of it is reduced.

Furthermore, a local anesthetic as described above may be used in order to counteract premature ejaculation in men.

The composition in accordance with the invention can be used for both heterosexual and also for homosexual men. Instead of transmitting pathogens in a vagina, this transmission can also be prevented in the case of other body orifices, for example the anus. In the case of same-sex sexual intercourse, the advantage lies in the gel nature of the form of application, which allows for smoother sexual intercourse and stops the transmission of sexually transmitted diseases. The gel nature of the composition means that microlesions of the lining of the anus are massively reduced, a frequently occurring problem with the use of conventional condoms, which can subsequently tend to rupture. By preventing microlesions of the lining, the risk of infection with pathogens is very substantially reduced.

The composition in accordance with the invention may be used or may be provided for a use in order to reduce any transmission of pathogens. Pathogens of this type may be bacteria, fungi or viruses. Examples of bacteria the transmission of which is inhibited in accordance with the invention are S. aureus, E. coli, P. aeruginosa. Examples of fungi the transmission of which is inhibited in accordance with the invention are C. albicans, S. cerevisiae. The antiviral action of polymeric guanidines and biguanidines has often been described in the literature (for example Klein et al., Journal of General Virology (2000), 81, 895-901).

The polymeric guanidine exhibits strong virucidal activity, in particular against naked viruses. The antiviral activity against conventional viruses which are transmitted during sexual intercourse and which should be protected against has been described in the literature, partly by tests with the structurally similar polymeric guanidine derivative polyhexamethylenebiguanide (PEYIB), see Romanowski et al. (JAMA Ophthalmol. 2013 April; 131 (4): 495-8), Gentile et al. (BMC Clinical Pathology 201212: 17), Valluri et al. (Cornea. 1997; 16 (5): 556-559), Passic et al. (Biomed Pharmacother. 2010; 64 (10): 723-732). The composition in accordance with the invention can reduce or prevent the transmission of viruses from the virus carrier to the cells of the contact person by means of the active substances and by the gel/acid/polymer mixture. Examples of viruses the transmission of which is reduced or prevented are HPV (Human Papilloma Virus), HSV (Herpes Simplex Virus) or HIV (Human Immunodeficiency Virus).

The invention can provide specific prevention of the transmission of an infection with these pathogens, for example after a sexual partner has been diagnosed, or a prophylactic use, for example in order to prevent possible transmission.

The invention will now be described in more detail by way of illustration by means of the following figures and examples.

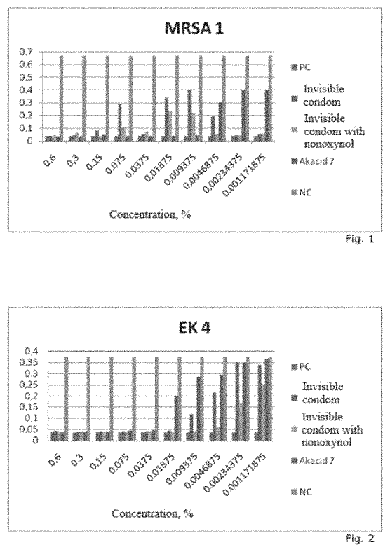

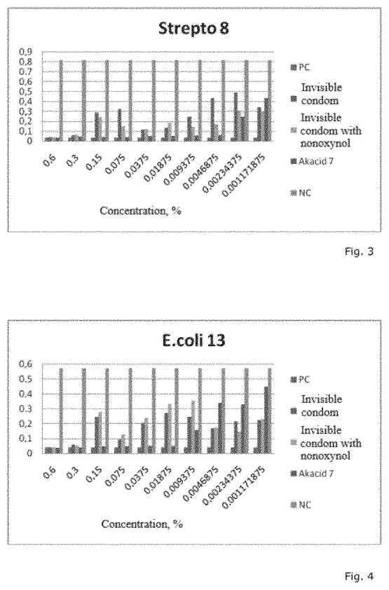

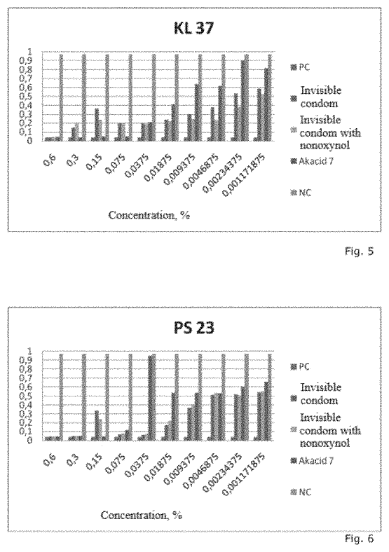

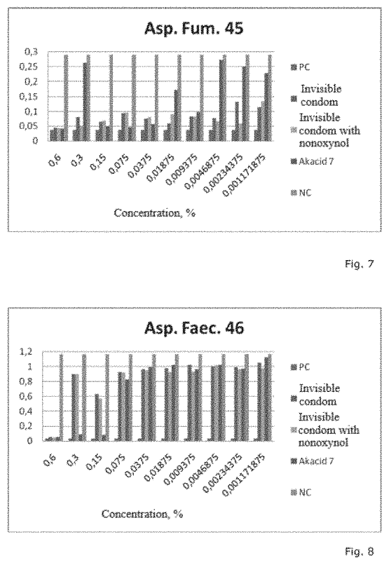

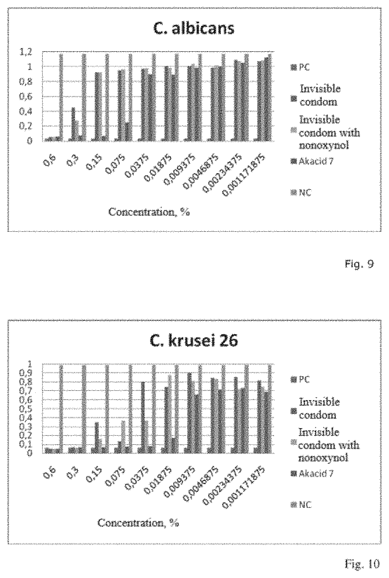

The figures show the inhibiting action of the composition in accordance with the invention and pure Akacid in different concentrations of the substance against various pathogens, namely against MRSA1 (FIG. 1), EK4 (FIG. 2), Strepto 8 (FIG. 3), E. coli 13 (FIG. 4), KL 37, (FIG. 5), PS23 (FIG. 6), Asp. fum. 45 (FIG 7), Asp. faec. 46 (FIG. 8), C. albicans (FIG. 9), C. krusei (FIG. 10), in each case together with positive controls (PC) and negative controls (NC).

### EXAMPLE 1

### Production of a Polyguanidine Formulation

The basic formulations were biocidal substances from the polyguanidine group (Akacid, described in Kratzer et al., Antibiotika Monitor Jan. 2, 2006; Buxbaum et al., Journal of Antimicrobial Chemotherapy (2006) 58,193-197; WO 01/85676 A1; WO 2006/047800 A1; WO 2008/080184 A2; WO 2013/064161 A1), in various formulations such as Akacid Plus (3:1 mixture of polyhexamethylene guanidinium chloride and poly-[2-(ethoxy)eyethyl]guanidinium chloride). These biocidal polymeric guanidine compounds have an excellent antimicrobial profile and in addition are capable of delivering stable formulations with substances which are difficult to mix without solubilizers from the group formed by polyethoxylated non-ionic compounds.

Akacid was produced in a modified method in which the starting substances tri ethylene glycol with hexamethylenediamine and guanidine hydrochloride were reacted in a 1-step synthesis and then freed from low molecular weight and high molecular weight monomers by filtration through three NF membranes so that a monomeric purified Akacid Plus fraction with. better pharmacological properties was produced. The mass average molecular weight of this Akacid Pius (or “X-Cid” was approximately 1000 Dalton (mainly 500 to 3000 Dalton). The composition was prepared as an aqueous solution for further formulation.

The quantities of the substances are given in g or as a %. All of the percentages are given as a % by weight, unless indicated otherwise.

### EXAMPLE 2

### “Liquid Condom Female” Formulation

The “liquid condom female” formulation consisted of a gel containing hydroxyethylcellulose. The hydroxyethylcellulose was expanded with Aqua Purificata for 5 minutes until a low viscosity gel was obtained.

A citric acid solution was incorporated into this gel base; the pH was adjusted to 5.5-6.2 by means of a sodium hydroxide solution after mixing in all of the other substances.

The two active substances Akacid Plus 1000 (in a concentration of 0.2-1%) and the spermicidal substance 9-nonoxinol (in a concentration of 5%) or other substances which promote contraception such as lactic acid, citric acid, quinine, pomegranate seed extract, honey or crushed acacia shoots were incorporated into the prepared gel base. Administration of the gel was carried out using 1.8 mL disposable plastic pipettes the head of which had to be twisted off before use. The total amount has to be inserted intravaginally at least 1-3 minutes prior to sexual intercourse. The gel is then distributed by the sexual activity itself.

Formulation I, “Liquid Condom Female”:

| Akacid Plus 1000, 50% | 0.8 g | |
|---|---|---|
| | (i.e. 0.4 g Akacid) | |
| Acidum Citricum | 1.5 | g |
| propylene glycol | 10.0 | g |
| nonoxynol-9 | 5.0 | g |
| hydroxyethylcellulose | 2.0 | g |
| sodium hydroxide | 0.75 | g |
| Aqua purificata | 79.95 | g |
| | 100.0 | g |

### EXAMPLE 3

### “Liquid Condom Male” Formulation

The “liquid condom male” formulation is a hydroxyethylcellulose-containing gel. The hydroxyethylcellulose was expanded with Aqua Purificata for 5 min. until a low viscosity gel was obtained with shearing. The two active substances Akacid Plus 1000 (for example in a concentration of 0.2%) and the local anesthetic lidocaine hydrochloricum (in a concentration of 0.4%) were incorporated into the ointment base which had been produced. The local anesthetic was added for the purposes of desensitization in order to counteract premature ejaculation. The pH here was neutral to slightly basic. The formulation did not have a contraceptive action and primarily serves to prevent infection of the male by otherwise unprotected sexual intercourse.

Formulation II “Liquid Condom Male”:

| Akaoid Plus 1000, 50% | 0.6 g | |
|---|---|---|
| | (i.e. 0.3 g Akacid) | |
| propylene glycol | 3.0 | g |
| hydroxyethylcellulose | 3.0 | g |
| lidocaine hydrochloricum | 0.4 | g |
| Aqua purificata | 93.0 | g |
| | 100.0 | g |

### EXAMPLE 4

### “Liquid Condom Suppository” Formulation

The weighed Akacid Plus 1000 was incorporated into the molten Adeps Neutralis solution together with the nonoxynol-9, and after cooling to approximately 40-45° C. was cast into 2 g suppository moulds and allowed to cool and solidify.

Formulation III “Liquid Condom Suppository 1%”, 10 Pieces:

| Akacid Plus 1000, 50% | 0.4 g | |
|---|---|---|
| | (i.e. 0.2 g Akacid) | |
| Adeps Neutralis | 19.0 | g |
| nonoxinol-9 | 1.6 | g |
| | 21.0 | g |

### EXAMPLE 5

### Comparison of Antibacterial Effect of the “Liquid Condom” Compared with Pure Akacid

The following compositions were tested by comparison with each other, wherein only the Akacid concentration (as Akacid Pius as described in Example 1) was varied, in a variety of dilutions.

Composition I: “liquid condom” as described in Example 2(“liquid condom female”) with nonoxinol-9

Composition 2: “liquid condom” as described in Example 2(“liquid condom female”) without nonoxinol-9(water instead)

Composition 3: Akacid Plus, remainder: water

Pathogens tested:
MRSA 1—Staphylococcus aureus
EK 4—Enterococcus taecium
Strepto 8—Streptococcus pneumoniae
E. coli 13—Escherichia coli
KL 37—Klebsiella pneumonia
PS 23—Pseudomonas aeruginosa
Asp. fum. 45—Aspergillus fumigatus
Ssp. faec. 46—Aspergillus fumigatus
C. alb.—Candida albicans
C. Krusei 26—Candida krusei

Media:

For bacteria: Müller Hinton Broth

For fungi: Sabouraud Broth

Assay:

50 μL of the respective medium was placed in all of the wells of 96-well plates, with the exception of the control wells, into which 100 μL of medium per well was added. Samples were prepared and 100 μL thereof was added to the wells.

50 μL of the test compositions was added to the wells, wherein the test compositions had different X-cid concentrations, starting from 0.6%, and respective 1:1 dilutions with the remaining composition components, i.e. 0.6%, 0.3%, 0.1%, 0.075%, 0.07%, 0.0188%, 0.00938%, 0.00469%, 0.00234%, 0.00117% (rounded, all percentages as % by weight).

Pathogen samples were thawed and diluted in the respective medium. 50 μL of the pathogen sample was added to all of the wells (apart from the negative control). Incubation was carried out overnight at 35° C. On the following day, the plates were visually assessed and measured using the SpectraMax 190 reader.

The results of the measurement with the SpectraMax 190 reader are shown in FIGS. 1-10.

Clearly, with MRSA there was a “liquid condom” activity in all of the concentrations. Individual outliers were assumed to be due to possible unequal quantities of material being transported with the pipette. This might be possible because the substance itself was very viscous, and thus it was sometimes difficult to pipette exact quantities.

This was also the case for the “liquid condom” with nonoxynol.

For Akacid 7 (=X-Cid test substance), an activity up to a concentration of 0.009375% could be observed. After this, no more inhibition could be obtained.

With EK 4, the “liquid condom” activity could be observed up to a concentration of 0.08175%, while the “liquid condom” activity in combination with nonoxynol could be observed up to a concentration of 0.009375%.

Akacid 7 was active up to a concentration of 0.0375%. Surprisingly, this means that the “liquid condom” was more effective than pure Akacid for the same Akacid concentration. This can only be assumed to be due to a synergistic effect of the composition.

With Streptococcus 8, variations in the visibility of the activity were again present, so that a true inhibition could only be obtained in the first two concentrations.

For Akacid 7, an activity against streptococcus 8 could be observed up to a concentration of 0.0046875%.

For E. coli 13, variations in the activity for the “liquid condom” as well as for the “liquid condom” in combination with nonoxynol were again observed. For Akacid 7, there was activity up to a concentration of 0.01875%.

For KL 37, the activity of the “liquid condom” was observed up to a concentration of 0.009375%, while activity of the “liquid condom” in combination with nonoxynol was observed up to a concentration of 0.0046875%. For Akacid 7, an activity of up to a concentration of 0.0375% could be observed.

For PS 23, the activity for Akacid 7 was up to a concentration of 0.0375%. For the “liquid condom” and the combination of “liquid condom” and nonoxynol, an inhibition of activity was observed up to a concentration of 0.01875%.

For Aspergillus fumigatus 45, the activity of the “liquid condom” and the combination of “liquid condom” with nonoxynol was observed in almost all concentrations (up to 0.00118%—minimum tested concentration). For Akacid 7, the activity was only obtained up to 0.009%. For Aspergillus fumigatus 46, the activity for the “liquid condom” as well as for the combination of “liquid condom” with nonoxynol was present at 0.6%. For Akacid 7, the activity for a concentration of 0.15% was observed. With Candida albicans, the activity for the “liquid condom” as well as for the “liquid condom” in combination with nonoxynol was 0.3%. For Akacid 7, the inhibition was visible up to a concentration of 0.075%.

For Candida krusei 26, the inhibition for Akacid was the highest compared with “liquid condoms” and the combination of “liquid condom” with nonoxynol, and in fact with an inhibition up to a concentration of 0.08175%. For the “liquid condom”, inhibition up to a concentration of 0.075% was visible. For “liquid condoms” in combination with nonoxynol, inhibition was observed up to a concentration of 0.0375%.

In summary, almost no difference or only a very small difference was observed between the “liquid condom” alone and “liquid condoms” with nonoxynol. However, in most cases, Akacid 7 exhibited a lesser inhibiting effect compared with the two “liquid condom” compositions.

1. A chemical composition containing at least one polymeric guanidine biocide and at least one alkylphenoxypolyethoxyethanol spermicide or a local anesthetic, in aqueous solution, and at least one thickening agent.

2. The composition as claimed in claim 1, characterized in that the polymeric guanidine biocide is a polyalkylene guanidine, preferably a polyoxyalkylene guanidine.

3. The composition as claimed in claim 2, characterized in that the polymeric guanidine biocide is selected from polyhexamethylene guanidine; poly[2-(2-ethoxy)ethoxyethyl guanidinine; polytriethyleneglycol guanidine; polyethyleneglycol guanidine; polyoxypropylene guanidine; polyoxyethylene guanidine.

4. The composition of claim 1, characterized in that the guanidine is a guanidinium salt, preferably selected from a halide, preferably a chloride; phosphate, preferably a dihydrogen phosphate; carbonate; nitrate; sorbate; acetate, preferably hydroacetate; gluconate, citrate, silicate.

5. The composition of claim 1, characterized in that the mean molecular weight of the polymeric guanidine biocide is 200 Da to 10000 Da, preferably 500 Da to 3000 Da.

6. The composition of claim 1, characterized in that the alkylphenoxypolyethoxyethanol spermicide is nonylphenoxypoly(ethyleneoxy) ethanol (nonoxynol-9).

7. The composition of claim 1, characterized in that the thickening agent is a gel forming agent, preferably selected from hydroxyalkyl cellulose, cellulose, hydroxyethyl cellulose, hydroxypropyl cellulose, hydroxypropylmethyl cellulose; or a suppository base, preferably selected from hard fat, a glycerol-gelatine mixture or soluble PEG, for example PEG 400/4000.

8. The composition of claim 1, characterized in that the composition comprises 0.05% to 10% (by weight) of polymeric guanidine biocide.

9. The composition of claim 1, characterized in that the composition comprises 0.05% to 8% (by weight) of alkylphenoxypolyethoxyethanol spermicide.

10. The composition of claim 1, characterized in that the composition comprises 1% to 95% (by weight) of thickening agent.

11. The composition of claim 1, characterized in that the composition is in a single dose form in a quantity of 1 mL to 5 mL, preferably in a disposable pipette.

12. The composition of claim 1, characterized in that the composition contains a humectant, preferably propylene glycol.

13. The composition of claim 1, characterized in that the composition contains the following:
polymeric guanidine biocide: 0.05% to 10%,
humectant: 5% to 15%,
alkylphenoxypolyethoxyethanol spermicide: 0.05% to 10%,
thickening agent: 1% to 10%,
all as a % by weight,
preferably further containing:
Acidum Ascorbicum 0% to 3%,
sodium hydroxide: 0% to 2%,
citric acid: 0% to 4%,
Aqua purificata: remainder to 100%,
all as a % by weight.

14. A method of disinfecting a vagina in a subject during sexual activity, comprising introducing into the vagina of the subject chemical composition containing at least one polymeric guanidine biocide in aqueous solution, and at least one thickening agent.

15. A method for preventing infection with a sexually transmittable disease or for contraception during sexual activity, comprising introducing a composition of claim 1 into a vaginal canal for the sexual activity.
